(19) United States
(12) Patent Application Publication
(10) Pub. No.: US 2024/0196976 A1
(43) Pub. Date: Jun. 20, 2024

(54) Liquid Jet Inhalation Device

(71) Applicant: JT International SA, Geneva (CH)

(72) Inventors: James Alan Mott, San Diego, CA (US); Simon Dodd, Albany, OR (US); Gianluca Gulli, Sesto San Giovanni (IT); Domenico Giusti, Caponago (IT); Bruce Michael Eckard, San Diego, CA (US); Jeffrey John Glassett, Escondido, CA (US); Joseph Winn Fiske, San Marcos, CA (US)

(73) Assignee: JT International SA, Geneva (CH)

(21) Appl. No.: 18/288,661

(22) PCT Filed: Apr. 5, 2022
(86) PCT No.: PCT/EP2022/058949

§ 371 Date: Oct. 27, 2023

(30) Foreign Application Priority Data
May 6, 2021 (EP) 21172477.8

(51) Int. Cl. A24F 40/46; A24F 40/10; A24F 40/485; A24F 40/51; A24F 40/57; A61M 15/06; H05B 3/26

(52) U.S. Cl. CPC A24F 40/46; A24F 40/10; A24F 40/485; A24F 40/51; A24F 40/57; H05B 3/265; A61M 15/06

(57) ABSTRACT

An inhalation device with at least one liquid jet device for producing drops of a liquid on demand, the liquid jet device has a fluid chamber, an ejection nozzle and a supply channel embedded in a substrate. The inhalation device further includes an air conduit and a mixing chamber in which air from said air conduit is mixed with the generated liquid drops. The air conduit includes at least two heating elements arranged to pre-heat the air guided by the air conduit from a respective air inlet orifice into the mixing chamber.

### TECHNICAL FIELD

The present invention relates to inhalation devices with at least one liquid jet device for producing drops of a liquid on demand. More particularly, the present invention relates to such inhalation devices in the form of electronic cigarettes, cigalikes, e-cigarettes, vapour inhalers and related devices.

Generally, with regard to any further details, the disclosure of the applicant's applications entitled “INHALATION DEVICE WITH AT LEAST ONE LIQUID JET DEVICE, CARTRIDGE FOR AN INHALATION DEVICE AND METHOD OF CONVEYING LIQUID DROPS IN AN INHALATION DEVICE”, “CONTAINER FOR AN INHALATION DEVICE WITH AT LEAST ONE LIQUID JET DEVICE, COMBINATION OF AT LEAST TWO CONTAINERS AND METHOD OF CONVEYING LIQUID TO AN INHALATION DEVICE”, and “LIQUID JET INHALATION DEVICE” filed on the same day are incorporated herein by means of the reference.

### BACKGROUND

In the arts there are several types and concepts for inhalation devices that serve a broad range of purposes including medical and therapeutic applications and also leisure and pleasure devices such as electronic cigarettes. Existing inhalation devices either change the phase of a fluid before inhalation with for example a wick and a coil so as to significantly raise the vapor temperature above human body temperature or deliver drops a room temperature by, for example, employing an ultrasonic mesh. In the above wick and coil system the vapor can be perceived as ‘warm’ by a user in the mouth, whereas in the ultrasonic mesh systems, the vapor is usually perceived as ‘cold’.

Such inhalation devices are oftentimes portable and pocket-size devices that can easily fit in the user's hand or can be handled by the user's fingers alone. In this way, the devices can be convenient for use and can be carried by a user for regular or emergency use. In the former case, a user can conveniently carry an inhalation device in the form of an e-cigarette to use and enjoy it whenever and wherever desired, whereas in the latter case a medical or therapeutic inhalation device may be ready to use when needed.

As such devices are handheld and compact, there is the risk that users may hold or operate the inhalation device incorrectly. For example, the user may touch or interfere with parts and elements of the inhalation device which may negatively affect the operation of the device or even the quality or properties of the aerosol to be generated by the device and to be ultimately inhaled by the user. One such element may be the orifice through which inlet air is sucked that is then mixed with the droplets of the vapour or aerosol. At the same time, such inhalation devices may employ heaters and vaporizers that may well depend on a steady or at least well-defined flow of air.

There is therefore a need for improved inhalation devices that not only ensure convenience in use and carriage, but also ensure and improve reliability and quality of operation. It is thus an object of the present invention to provide such improved inhalation devices that can remedy the drawbacks of the conventional solutions.

### SUMMARY

The mentioned drawbacks are remedies by the subject-matter of the independent claims. Further preferred embodiments of the present invention are defined in the dependent claims.

According to one embodiment of the present invention there is provided an inhalation device with at least one liquid jet device for producing drops of a liquid on demand, said liquid jet device comprising a fluid chamber, an ejection nozzle and a supply channel embedded in a substrate, the inhalation device further comprising an air conduit and a mixing chamber in which air from said air conduit is mixed with the generated liquid drops, wherein the air conduit comprises at least two heating elements arranged to pre-heat the air guided by the air conduit respective air inlet orifice into the mixing chamber.

### BRIEF DESCRIPTION OF THE DRAWINGS

Embodiments of the present invention, which are presented for better understanding the inventive concepts and which are not to be seen as limiting the invention, will now be described with reference to the Figures in which:

FIGS. 1A to 1D show schematic views of an inhalation device according to respective embodiments of the present invention;

FIGS. 2A and 2B show schematic views of vapour generator assemblies of an inhalation device according to respective embodiments of the present invention;

FIG. 3 shows a schematic view of liquid jet device as employed in an inhalation device according to an embodiment of the present invention;

FIG. 4A shows a schematic view of an orifice configuration according to an embodiment of the present invention;
and

FIG. 4B shows a schematic view of a MEMS layout in a substrate according to an embodiment of the present invention.

### DETAILED DESCRIPTION

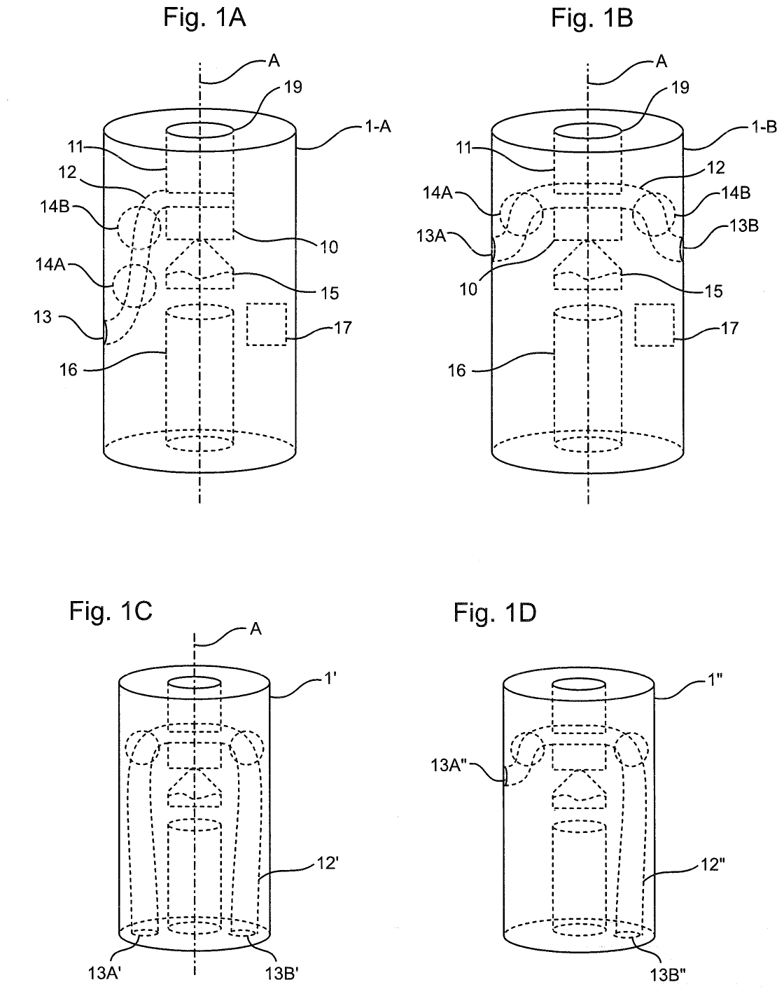

FIGS. 1A to 1D show schematic views of an inhalation device according to respective embodiments of the present invention. In FIG. 1A there is shown an inhalation device 1-A that comprises a liquid jet device 10 for producing drops of a liquid on demand. The liquid jet device 10 comprises a fluid chamber, an ejection nozzle and a supply channel embedded in a substrate, which details are shown and described elsewhere in the present disclosure. The term fluid chamber is meant to cover jet technologies generally, including at least piezo jet and thermal jet devices, wherein in the latter case the fluid chamber is then usually referred to as a firing chamber.

The inhalation device 1-A further comprises an air conduit 12 and a mixing chamber 11 in which air from said air conduit 12 is mixed with the liquid drops generated by the liquid jet device 10. The air conduit 12 further comprises at least one air inlet orifice 13 at some suitable site of said inhalation device 1-A. There are further at least two heating elements 14A, 14B arranged to pre-heat the air guided by the air conduit from the respective air inlet orifice into the mixing chamber 11. The inhalation device 1-A further comprises a mouthpiece opening 19 through which a user may inhale the inhalation vapour. The mouthpiece may be integral with the housing of the inhalation device 1-A, it may be replaceable, or may form part of a capsule or cartridge. The latter may comprise further elements, such as the mixing chamber, the liquid jet device or the reservoir so as to provide a replaceability of further elements for achieving convenience, flexibility, reliability and/or safety.

The inhalation device 1-A may further comprise a reservoir 15 for storing an amount of said liquid to be vaporized, a power source 16 in the exemplary form of a battery or a rechargeable battery, and a controller 17 that is configured to control all necessary parts and functions of the inhalation device 1. Liquid reservoirs, power sources and controllers (apart from the specific control program employed in the embodiments of the present invention) as from are such available the conventional arts, so that greater details of these elements are omitted here. Preferably, the inhalation device may comprise a reservoir configured to store an amount of said liquid and a reservoir heating element arranged to heat the liquid in said reservoir to a predetermined liquid reservoir temperature.

This is therefore an embodiment for an inhalation device with at least one liquid jet device for producing drops of a liquid on demand, said liquid jet device comprising a fluid chamber, an ejection nozzle and a supply channel embedded in a substrate, the inhalation device further comprising an air conduit and a mixing chamber in which air from said air conduit is mixed with the generated liquid drops, wherein the air conduit comprises at least two heating elements arranged to pre-heat the air guided by the air conduit from a respective air inlet orifice into the mixing chamber.

In an embodiment, the inhalation device 1-A may comprise a control unit 17, which may be configured to control the at least two heating elements 14A, 14B individually. In one embodiment, the at least two heating elements 14A, 14B may be arranged in series and to pre-heat the air guided by the air conduit 12 sequentially. In one embodiment, the at least two heating elements 14A, 14B may provide a different nominal heating power. In the case of a control unit 17 being present, this may be configured to control the at least two heating elements individually, in a simple on-and-off fashion.

This “digital” control of a heating element may provide the advantage that no linear, and hence resistive, or otherwise more or less complex, e.g. using pulse width modulation, power control is necessary. Power control can in this way still achieved, by simply energizing only one of the heaters or both heaters. In case, the heaters provide identical or similar nominal power P, a simple power control can already achieve three power levels 0 P, 1 P and 2 P by simply activating none, one or both heaters. In case, the heaters provide different powers Pa and Pb, a simple power control can already achieve four power levels including 0 P, Pa, Pb and Pa+Pb by simply activating none, one or both heaters.

Further, having two heaters may have the general benefit of twice the heating power of one sole heater. Generally, more heaters may produce either more volume of warm air or air that is warmer for a given volume. In a system where the heaters run at the same time, the total amount of air that can be heated can be increased. This could be accompanied by operating two heaters in parallel with potentially also individual inlet take orifices to more air in. In the alternative or additional system of putting heaters in series, the first heater could take the temperature from X to Y and the second heater could take the Y temperature air and move it to Z temperature where Y is greater than X and Z is greater than Y.

Having multiple heaters may also allow for a more efficient energy consumption to heat the air. It is possible that one large heater requires some amount P of power to raise the temperature by a given delta dT. If the heaters are based on convection for the heating element (e.g. wire) to air heat transfer then two heaters may have a larger amount of heating element in contact with the air than one heater. This could promote a more efficient heat transfer where two heaters can raise the air temperature by the same dT by using only P′<P power. Further, multiple heaters can be smaller than a single large heater. This means that multiple heaters may be able to be placed closer to the mouthpiece or they could be populated in cavities of the device that were previously unused. Multiple heaters provide design flexibility in placement of the components.

In FIG. 1B there is shown an inhalation device 1-B that comprises a liquid jet device 10 for producing drops of a liquid on demand. The liquid jet device 10 comprises a fluid chamber, an ejection nozzle and a supply channel embedded in a substrate, which details are shown and described elsewhere in the present disclosure. The term fluid chamber is meant to cover jet technologies generally, including at least piezo jet and thermal jet devices, wherein in the latter case the fluid chamber is then usually referred to as a firing chamber.

The inhalation device 1-B further comprises an air conduit 12 and a mixing chamber 11 in which air from said air conduit 12 is mixed with the liquid drops generated by the liquid jet device 10. The air conduit 11 further comprises at least two air inlet orifices 13A, 13B at different sites of said inhalation device 10 and for each inlet orifice 13A, 13B at least one heating element 14A, 14B arranged to pre-heat the air guided by the air conduit from the respective air inlet orifice into the mixing chamber 11. The inhalation device 1 further comprises a mouthpiece opening 19 through which a user may inhale the inhalation vapour. The mouthpiece may be integral with the housing of the inhalation device 1-B, it may be replaceable, or may form part of a capsule or cartridge. The latter may comprise further elements, such as the mixing chamber, the liquid jet device or the reservoir so as to provide a replaceability of further elements for achieving convenience, flexibility, reliability and/or safety.

The inhalation device 1 may further comprise a reservoir 15 for storing an amount of said liquid to be vaporized, a power source 16 in the exemplary form of a battery or a rechargeable battery, and a controller 17 that is configured to control all necessary parts and functions of the inhalation device 1-B. Liquid reservoirs, power sources and controllers (apart from the specific control program employed in the embodiments of the present invention) such from are as available the conventional arts, so that greater details of these elements are omitted here. Preferably, the inhalation device may comprise a reservoir configured to store an amount of said liquid and a reservoir heating element arranged to heat the liquid in said reservoir to a predetermined liquid reservoir temperature.

In embodiments of the inhalation device comprising a control unit, the unit 17 may be configured to control the at least two heating elements 14A, 14B individually. Specifically, the inhalation device may further comprise a pressure sensor for each inlet orifice 13A, 13B, wherein the control unit is configured to control the at least two heating elements 14A, 14B individually based on a respective output of said pressure sensors. In this way, the control unit 17 may be configured to immediately stop operation of one or more heaters when an output of the respective pressure sensor indicated an obstructed air flow. This may prevent not only damage to the inhalation device 1-B but may also reliably avoid a drop in the quality of the inhalation vapour. Pressure sensor (s) can thus be associated to the individual heated air-flow paths, and, with this, to individual heaters. This can provide the basis for a closed loop control for controlling the heaters and paths.

In the embodiment as shown in FIG. 1B, the at least two air inlet orifices 13A, 13B are preferably configured and arranged symmetrically relative to an axis A in the inhalation device 1-B. Preferably, the at least two air inlet orifices 13A, 13B are arranged on opposing sides of the inhalation device 1. FIGS. 1C and 1D show schematic views of further inhalation devices 1′ and 1″ according to further embodiments of the present invention. These embodiments consider similar or identical elements and functions as the one described in conjunction with FIG. 1B, but do provide different configuration with regard to the location of the orifices 13 relative to the inhalation device as such. In FIG. 1C, the two orifices 13A′, 13B′ are both located at a distal end of the inhalation device 1′, wherein in FIG. 1D, one orifice 13A″ is arranged at side of the inhalation device 1″, and another orifice 13B″ is arranged at a distal end of the inhalation device 1″. It is at this point noted that the embodiments described in conjunction with all FIGS. 1A to 1D can also be combined. For example, there may be more than one heater for one or each one of more than one path and orifice. Further, it is clear that the arrangement of orifices can be combined with any number of heaters.

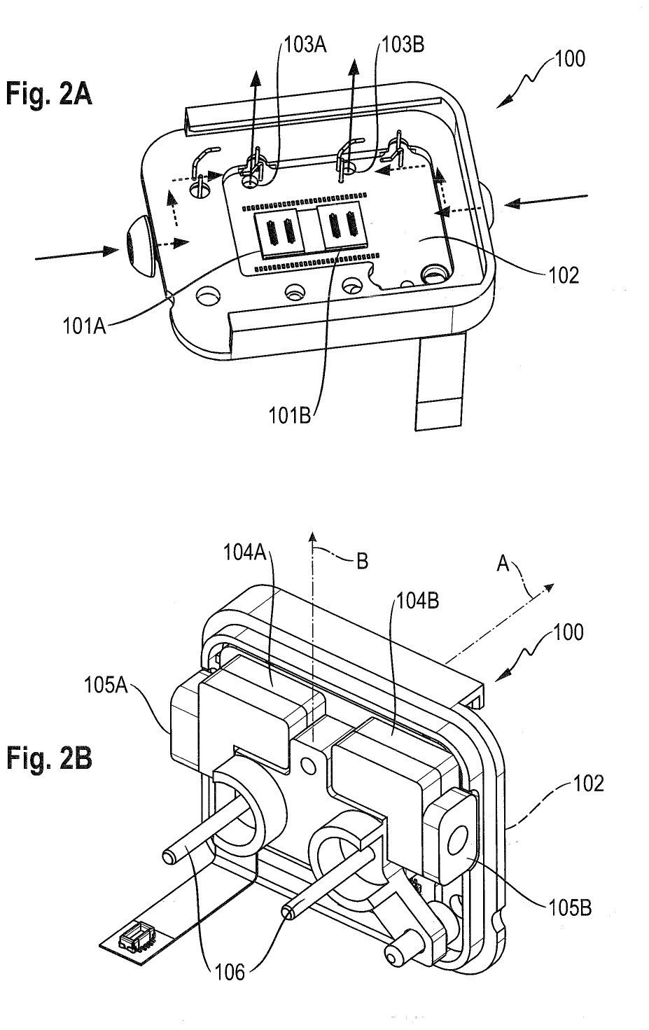

FIGS. 2A and 2B show schematic views of a vapour generator assembly 100 of an inhalation device according to respective embodiments of the present invention. In FIG. 2A there is shown the vapour generator assembly 100 from a front view from a side of the mixing chamber. As can be seen, there are two liquid jet device 101A, 101B in the form of micro-electromechanical systems (MEMSs) on a circuit board 102, wherein the pre-heated air is guided through opening 103A, 103B of said printed circuit board 102. The mixing chamber may be formed by placing an airtight cover on the circuit board which then acts as a rear limitation of the chamber, in which the air penetrating through the inlet opening 103A, 103B can mix with the drops generated by the liquid jet devices 101A, 101B. The cover may provide an outlet toward a mouthpiece or toward a user for inhalation.

In such a configuration the control, for example implemented by the already mentioned control unit 17, may comprise controlling a temperature of the drops. For such a purpose, the inhalation device may further comprise at least one further heating element arranged to pre-heat said liquid to a predetermined temperature prior to ejection through said ejection nozzle. For example, the further heating element can be a resistor embedded in a substrate of said MEMS so as to pre-heat to the predetermined temperature at least a part of the liquid present in the substrate. In principle, any resistor can be used as both power delivery to the device and measurement of resistance on the device. A resistor can thus for example increase the fluid temperature before ejection in a piezo-type jet device.

In an embodiment, such a resistor can be a temperature sense resistor, TSR, embedded in the substrate. Further, such a resistor can be a heating resistor arranged in a vicinity of said fluid chamber and configured to heat a first amount of the liquid to at least a vaporization temperature, so that a vapour bubble expels a drop of the liquid through the ejection nozzle. A TSR can be structured to be relatively long and narrow, maximizing the number of squares to maximize the sensitivity of the resistance measurement, which can be calibrated to correspond to a temperature. Therefore, the TSR can measure temperature and deliver heat to the substrate (silicon), which in turn can heat the fluid.

However, it is envisaged that the TSR would not heat the fluid to a vaporization temperature. If the substrate would get that hot there would be uncontrolled global ejection of drops. The TSR would heat the fluid to a maximum of ˜20° C. below the vapor temperature of the fluid. Then, in a thermal jet device, the heater in the fluid chamber would superheat the fluid to create the vapor bubble to eject the drops. In a piezo device the temperature constraints would be similar. But, once the fluid is in the fluid chamber the piezo actuator mechanically ejects the warm drop instead of creating a vapor bubble.

In FIG. 2B there is shown the vapour generator assembly 100 from a rear view from a side underneath the rear limitation of of the mixing chamber (e.g. in the form of a printed circuit board). In this embodiment there may be provided at least two heating elements 104A, 104B, one each for the inlet orifices 105A, 105B. The heating elements 104A, 104B may be configured and arranged symmetrically relative to an axis A or B in the inhalation device. Preferably, a heating element comprises a curved air flow path or even a meandering air flow path, so as to ensure sufficient and reliable heat exchange. In some embodiments the heating elements may comprise a ceramic heater housing. In other words, the airpath can be heated up before reaching the drop on demand generator. Air enters into the device through an orifice from which the air passes over one or more heating coils to provide convection heating of the air. The warm air enters the mouthpiece chamber where it is combined with the drops, preheated or at room/ambient temperature. This vapor mixture enters the mouth above ambient temperature.

In addition to the above, fluid intake channels 106, for example in the form of thin tubes or hollow needles, may extend downward from underneath the liquid jet devices mounted on the printed circuit board. These channels 106 may be in fluid communication with one or more reservoirs for storing one or more liquids as a base substance for vapor generation. In this way, more that one base substance can be employed to generate an adjustable and controllable mixture of several agents, flavours, etc.

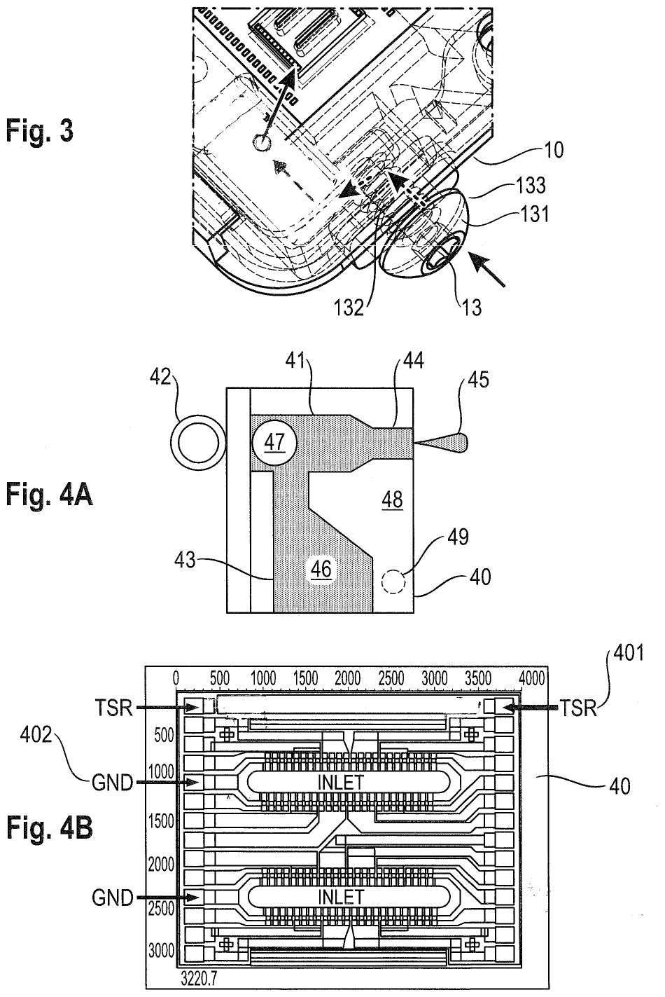

FIG. 3 shows a schematic view of an orifice configuration according to an embodiment of the present invention. The orifice 13 may be formed in an orifice element 131 that may comprise a tapped and stainless opening toward the external environment. The element 131 may be formed of resilient material that can be fitted into hole 132 in the inhalation device 10 so as to be adjustable and/or replaceable. The outer part of the orifice element 131 may be designed to avoid obstruction and/or adhesion of dust and dirt particles. For example, a resilient material and a collar 133 may render it difficult to obstruct the orifice 13 when force is applied, for example by a user's finger or part of a hand, to the element 131, as this may as it may give way to the force but still provides air throughput.

FIG. 4A shows a schematic view of liquid jet device as employed in an inhalation device according to an embodiment of the present invention. The liquid jet device 40 is formed as a MEMS in a substrate 48 of any suitable material, for example silicon. In that substrate 48 there is formed a fluid chamber 41, an ejection nozzle 44, and a supply channel 43 in liquid communication with a reservoir for providing liquid 46 to the fluid chamber 41 so as to be vaporized or atomized. A heating element 42 is arranged in the vicinity of the fluid chamber 41 so as to heat up a portion of the liquid 46 to vaporized and form a gas bubble 47. The resulting expansion leads to the ejection of an amount of the liquid in the form of a drop or droplet 45 that then can form in the mixing chamber a vapour or aerosol.

In some embodiments, there may be provided a further heating element 49 arranged to pre-heat said liquid 46 to a predetermined temperature prior to ejection through said ejection nozzle 44. For example, the further heating element 49 can be a resistor embedded in the substrate 48 so as to pre-heat to the predetermined temperature at least a part of the liquid 46 present in the fluid chamber 41 or supply channel 43. In an embodiment, such a resistor can be a temperature sense resistor, TSR, embedded in the substrate. The temperature of the liquid can be raised just before being ejected.

Here, the TSR is used for heating, in which a MEMS die is provided with a TSR trace to measure the temperature of the substrate (e.g. silicon) and, in steady state, the temperature of the liquid being ejected. This can be a trace with high current carrying capability. By putting voltage to the TSR, heat can be generated through resistance. With enough voltage and time, the temperature of the die can be raised to 250° C.

However, such elevated temperature may be desirable for some applications, they may be avoided and may be maintained below 150° C. at which MEMS polymer layers in the fluid chamber and nozzle may start to melt. Further, if water was being ejected, at temperatures around 95° C.-100° C. drops would start to shoot out of the fluid chamber in an uncontrolled manner. So, in general, the liquid in the assembly can be heated to a similar temperature as the die, wherein an ejecting a ‘warm’ drop of fluid can also create a warmer vapor for inhalation. FIG. 4B shows a schematic view of a MEMS layout in a substrate according to an embodiment of the present invention. There is specifically shown an exemplary position of the TSR 401 on a MEMS device 40. By means of the TSR solution, there can be obtained a reduction on the particle/droplet size. Especially, when the air temperature is enough there can be vaporized small droplets in the size range of <8 μm. By use of the liquid jet device technology there can be generated small droplets, and, in turn, the benefit would be obtained of smaller droplet sizes due to the vaporization of those.

Further, this purpose and functionality may also be combined with heating resistor 42 arranged in a vicinity of said fluid chamber 41 and configured to heat a first amount of the liquid 46 to at least a vaporization temperature, so that a vapour bubble 47 expels a drop 45 of the liquid 46 through the ejection nozzle 44. For example, the resistor 42 may be driven to heat the fluid chamber 41 and/or the supply channel 43 to a temperature below a threshold that would result in forming the bubble 47 and, consequently, in expelling the drop/droplet 45. Yet still, the relevant part of the liquid 46 could be at a predetermined temperature prior to ejection. In a way, this can be named as pulse warming by providing a pulse that is for example half the time as a firing pulse, wherein the heater warms up significantly but not enough to eject droplets. Through thermal conduction the liquid in the chamber can thus be increased. When this liquid is ejected also the aerosol or vapor temperature is increased for inhalation.

Although detailed embodiments have been described, these only serve to provide a better understanding of the invention defined by the independent claims and are not to be seen as limiting.

1. An inhalation device with at least one liquid jet device for producing drops of a liquid on demand,
said liquid jet device comprising a fluid chamber, an ejection nozzle and a supply channel embedded in a substrate,
the inhalation device further comprising an air conduit and a mixing chamber in which air from said air conduit is configured to be mixed with the generated liquid drops,
wherein the air conduit comprises at least first and second heating elements arranged to pre-heat the air guided by the air conduit from a respective air inlet orifice into the mixing chamber.

2. The inhalation device according to claim 1, wherein the at least first and second heating elements are arranged in series and to pre-heat the air guided by the air conduit sequentially.

3. The inhalation device according to claim 1, wherein the at least first and second heating elements provide a different nominal heating power.

4. The inhalation device according to claim 1, further comprising a control unit configured to control the at least first and second heating elements individually.

5. The inhalation device according to claim 1, wherein one of the at least first and second heating elements comprises a curved air flow path.

6. The inhalation device according to claim 5, wherein one of the at least first and second heating elements comprises a meandering air flow path.

7. The inhalation device according to claim 1, wherein one of the at least first and second heating elements comprises a ceramic heater housing.

8. The inhalation device according to claim 1, wherein the air conduit comprises at least first and second air inlet orifices at different sites of said inhalation device and for each of the at least first and second inlet orifice at least one of the at least first and second heating elements arranged to pre-heat the air guided by the air conduit from the respective air inlet orifice into the mixing chamber.

9. The inhalation device according to claim 8, wherein the at least first and second air inlet orifices are configured and arranged symmetrically relative to an axis in the inhalation device.

10. The inhalation device according to claim 8, wherein the at least first and second air inlet orifices are arranged on opposing sides of the inhalation device.

11. The inhalation device according to claim 8, further comprising a pressure sensor for each of the at least first and second inlet orifices, and wherein said control unit is configured to control the at least first and second heating elements individually based on a respective output of said pressure sensors.

12. The inhalation device according to claim 1, wherein the liquid jet device is in the form of a micro-electromechanical system (“MEMS”).

13. The inhalation device according to claim 12, further comprising at least one third heating element arranged to pre-heat said liquid to a predetermined temperature prior to ejection through said ejection nozzle.

14. The inhalation device according to claim 13, wherein the at least third heating element is a resistor embedded in a substrate of said MEMS so as to pre-heat to the predetermined temperature at least a part of the liquid present in the substrate.

15. The inhalation device according to claim 14, wherein the resistor is a temperature sense resistor (“TSR”) embedded in the substrate.

16. The inhalation device according to claim 14, wherein the resistor is a heating resistor arranged in a vicinity of said fluid chamber and configured to heat a first amount of the liquid to at least a vaporization temperature, so that a vapour bubble expels a drop of the liquid through the ejection nozzle.

17. The inhalation device according to claim 1, wherein a MEMS liquid jet device is mounted on a printed circuit board, and the pre-heated air is configured to be guided through an opening of said printed circuit board.

18. The inhalation device according to claim 17, the MEMS liquid jet device comprises at least two MEMS liquid jet devices mounted on said printed circuit board.

19. The inhalation device according to claim 18, further comprising a control unit for controlling a temperature of the drops.

20. The inhalation device according to claim 1, further comprising a reservoir configured to store an amount of said liquid and a reservoir heating element arranged to heat the liquid in said reservoir to a predetermined liquid reservoir temperature.
